(19) United States
(12) Patent Application Publication
(10) Pub. No.: US 2025/0051487 A1
(43) Pub. Date: Feb. 13, 2025

(54) HYALURONIC ACID DERIVATIVE HAVING A REDUCED POLARITY, METHOD OF PREPARATION THEREOF, COMPOSITION AND USE THEREOF

(71) Applicant: CONTIPRO A.S., Dolni Dobrouc (CZ)

(72) Inventors: Radovan Buffa, Humenne (SK); Vit Svozil, Usti nad Orlici (CZ); Tomas Klejch, Litomysl (CZ); Martina Hermannova, Zamberk (CZ); Lenka Hejlova, Vyprachtice (CZ); Hana Vagnerova, Dolni Cermna (CZ); Kristina Nesporova, Usti nad Orlici (CZ); Lenka Nespechalova, Vamberk (CZ); Martina Brandejsova, Letohrad (CZ); Eva Krivakova, Sokol (SK); Vladimir Velebny, Zamberk (CZ)

(73) Assignee: CONTIPRO A.S., Dolni Dobrouc (CZ)

(21) Appl. No.: 18/719,598

(22) PCT Filed: Dec. 14, 2022
(86) PCT No.: PCT/CZ2022/050131

§ 371 Date: Jun. 13, 2024

(30) Foreign Application Priority Data
Dec. 14, 2021 (CZ) PV2021-563

(51) Int. Cl. C08B 37/08; A61K 8/73; A61K 31/728; A61Q 19/08

(52) U.S. Cl. CPC C08B 37/0072; A61K 8/735; A61K 31/728; A61Q 19/08

(57) ABSTRACT

The invention relates to a method of preparation of hyaluronic acid derivative, that has some carboxyl groups (—COOH) replaced by the primary hydroxyl group (—CH₂—OH). The method of preparation is based on the carboxyl group activation using 4-(4,6-dimethoxy-1,3,5-triazine-2-yl)-4-methylmorpholinium chloride agent and subsequent reduction with NaBH₄. The hyaluronic acid derivatives prepared according to invention are biocompatible and biodegradable, showing increased resistance to thermal and enzymatic degradation. Formula for annotation (I):

I═R¹-(A)_{X}-(B)_{Y}—R²
wherein
A is a structural fragment of a linear chain:

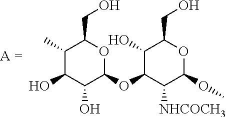

B is a structural fragment of a linear chain:

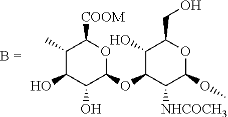

R¹ is the structural fragment of the end of the linear chain:

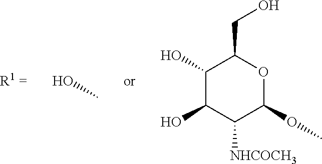

R² is the structural fragment of the end of the linear chain:

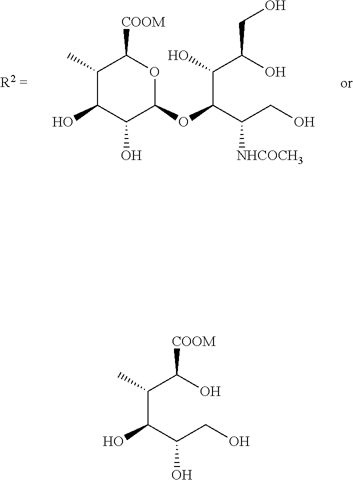

M can be hydrogen or any alkali metal cation,
X is an index indicating the number of structural fragments A within the chain,
Y is an index indicating the number of structural fragments B within the chain, while at the same time:
   structural fragments A and B are randomly distributed in the linear chain, and
   the sum of the X+Y indexes is in the range of 500 to 2,500, and
   the ratio of the X/Y indexes is in the range of 3/1 to 1/4.

### TECHNICAL FIELD

The invention refers to a reduced hyaluronic acid derivative or its salt in the form of a linear chain having a reduced polarity, which is caused by modification of the carboxyl group to primary alcohol according to structural formula I:

I═R¹-(A)_{X}-(B)_{Y}—R²
wherein A is a structural fragment of a linear chain:

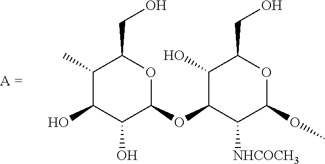

B is a structural fragment of a linear chain:

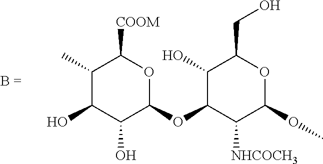

R¹ is a structural fragment of the end of a linear polymer:

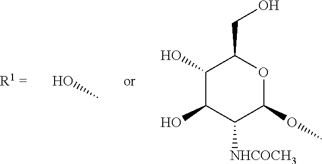

R² is a structural fragment of the end of a linear polymer:

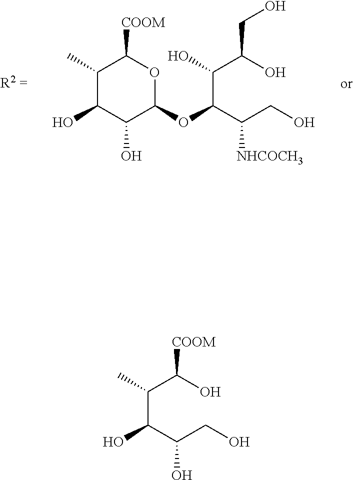

M can be hydrogen, any metal cation, such as alkali metal cation,
X is an index indicating the number of structural fragments A within the chain,
Y is an index indicating the number of structural fragments B within the chain, while at the same time:
   structural fragments A and B are randomly distributed in the linear chain, and
   the sum of the X+Y indexes is in the range of 500 to 2,500, and
   the ratio of the indexes X/Y is in the range of 3/1 to 1/4.

The polysaccharide thus modified shows increased resistance to thermal and enzymatic degradation while maintaining the safety and biocompatibility typical for the native hyaluronic acid.

Furthermore, the invention relates to a method of preparation of the polysaccharide according to the structural formula I, wherein the modification of hyaluronic acid is made using 4-(4,6-dimethoxy-1,3,5-triazine-2-yl)-4-methylmorpholinium chloride (DMTMM) and sodium borohydride (NaBH₄) in a mixture of water/acetonitrile.

Furthermore, the invention relates to the use of the polysaccharide according to the structural formula I in areas where the following characteristics are required, or a combination thereof:
use of biocompatible and biodegradable polysaccharide
increased resistance to thermal degradation compared to the native hyaluronic acid
increased resistance to enzymatic degradation compared to the native hyaluronic acid
possibility to regulate the rate of degradation
increased viscosity of solutions
reduced swelling of solution or solid forms

### State of the Art

### Hyaluronic Acid

Hyaluronic acid or its sodium salt is a non-sulfated glycosaminoglycan composed of two repeating units of D-glucuronic acid and N-acetyl-D-glucosamine (Formula 1).

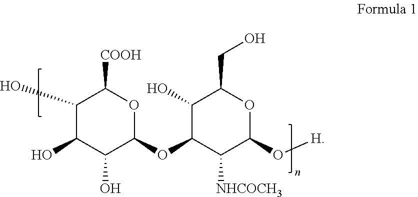

The molecular weight of the native hyaluronic acid can reach up to 5,000 kg·mol⁻¹ in the human body. This polysaccharide forms an important part of the connective tissues, skin and synovial fluid of the joints and plays an important role in a number of biological processes such as hydration, cell differentiation and the proteoglycans organization. Hyaluronic acid occurs naturally in biological systems, so it is naturally biodegradable and biocompatible. Therefore, it is a suitable substrate for a wide range of biomedical applications.

The following chapters describe procedures that modify carboxyl groups in different types of substrates (low molecular weight substances to polysaccharides) in protic, aprotic or solvent-free conditions, where in some cases the configuration on the carbon next to the carboxyl group is inverted.

From the point of view of the possible use of reaction conditions for the reduction of hyaluronic acid to the derivative of the general formula I, it is important to consider:
suitability of the solvent for dissolving hyaluronic acid
the possibility that the reaction conditions will cause, in addition to carboxyl reduction, significant degradation of the hyaluronic acid molecular weight
possibility that the reaction conditions will cause other hyaluronic acid modifications in addition to carboxyl reduction (configuration inversions, reactions on hydroxyl or amide groups)
   Reduction of Carboxylic Acids with Boranes:

Effective reduction of low molecular weight aliphatic and aromatic carboxylic acids to the respective alcohols can be achieved with an excess (3-4 equivalents) of pinacol borane at 20° C. without solvents and catalyst (Harinath A. et al: Chem. Comm., 10, 55, 1386-9, 2019). However, this approach cannot be applied to molecules containing a large number of hydroxyl groups that would react rapidly with borane to form hydrogen.

Simple aliphatic alpha-hydroxy carboxylic acids can be reduced in tetrahydrofuran using a system (NaBH₄+I₂) that is expected to form diborane B₂H₆ or its analogues (Burgess K. et al.: Tetrahedron Letters, 36, 16, 2725-2728, 1995).

The patent publication (Getman D. P. et al.: U.S. Pat. No. 6,388,132, 2002) described selective reduction of aliphatic carboxylic acids, which contain a carbamate group in their structure. The reduction to alcohol proceeds with borane in tetrahydrofuran.

Selective reduction of aliphatic carboxylic acids, which contain hydroxyl groups and ethers in their structure has been described in the article (Murray J. et al: Chem. Comm., 50, 88, 13608-13611, 2014). The reaction proceeds for 12 hours at 0 to 60° C. in an inert atmosphere and the system (NaBH₄+I₂) in tetrahydrofuran was used as the reduction reagent.

The same reagent (NaBH₄+I₂) was used for selective reduction of aliphatic carboxylic acids, which contain hydroxyl groups and lactones in their structure (CN 110845454, 2020). The reaction proceeds for 2 hours in tetrahydrofuran at 25° C.

The reduction of aliphatic carboxylic acids, which contain ether groups in their structure, was described in the article (Kano S. et al: Synthesis, 9, 695-697, 1980). Boranes generated by the system (NaBH₄+TiCl₄) were used as reagents. The reaction proceeds for 14 hours in 1,2-dimethoxyethane at 20° C.

The use of boranes generated by the system (NaBH₄+H₂SO₄) to reduce aliphatic carboxylic acids containing ether groups was described in the patent publication (Haldar P. et al.: US250454, 2011). The reaction proceeds for 45 minutes in tetrahydroforane and diethyl ether in the temperature range from 0 to 35° C.

The reduction of aliphatic carboxylic acids, which contain acetyl and other ester groups in their structure, can be achieved using the dimethyl sulfide/borane complex in tetrahydrofuran (Williams S. J. et al.: Journal of the American Chemical Society, 122, 10, 2223-2235, 2000). The reaction proceeds at 20° C. with a longer reaction time required (up to 72 hours).

In general, when reducing carboxylic acids either directly with boranes, or boranes complexed with dimethyl sulfide, or with boranes generated in situ, the use of protic solvents is inappropriate due to the rapid reaction of boranes with the hydrogen of the solvent and the formation of the molecular hydrogen. These reactions proceed efficiently either in aprotic systems (ethers) or without the solvent presence.

Reduction of Carboxylic Acids with Aluminum Hydrides.

The reduction of aliphatic carboxylic acids, which contain ester groups in their structure, can be achieved by two-step synthesis, where in the first step the carboxyl group is activated by oxalyl dichloride in N,N-dimethylformamide, tetrahydrofuran, dichloromethane and acetonitrile at −78 to +20° C., and in the second step the activated carboxyl group is reduced with lithium tri-(tert-butoxy)aluminum hydride in tetrahydrofuran, dichloromethane and acetonitrile at 20° C. within 1.5 hours (Chany A. C. et al.: Organic and Biomolecular Chemistry, 13, 35, 9190-9193, 2015).

The reduction of hyaluronic acid hexasaccharide with a modified carboxyl to methyl ester was described in the article (Onoera K. et al.: Agricultural and Biological Chem., 27, 2, 143-149, 1963). LiAlH₄ in tetrahydrofuran was used as reagent.

Even in the reduction of carboxylic acids with aluminum-based hydrides, the use of protic solvents is inappropriate due to the rapid reaction of aluminum hydrides with the hydrogen from the solvent to form molecular hydrogen. Reductions proceed efficiently in aprotic solvents such as tetrahydrofuran, dichloromethane or acetonitrile.

Reduction of Carboxylic Acids with Boron Hydrides

The reduction of aliphatic carboxylic acids, which contain primary amines and hydroxyl groups in their structure, can be achieved by activation of carboxyl with chlorotrimethylsilane and subsequent reduction with lithium borohydride in tetrahydrofuran under an inert atmosphere (Jing, Q. et al.: Bioorganic and Medicinal Chemistry Letters, 23, 20, 5674-5679, 2013).

In the article (Montchamp J.-L. et al.: Journal of the American Chemical Society, 114, 12, 4453, 1992) reduction of aliphatic carboxylic acids, which also contain hydroxyl groups and ethers in their structure, was described by activating carboxyl with triethyl orthoformate and 4-methylmorpholine-N-oxide. The subsequent reduction was made using NaBH₄ with osmium oxide in ethanol and water. Racemisation in the alpha carboxyl position was observed as by-product.

NaBH₄ was used to reduce a wide range of carboxylic acids after in situ activation with benzotriazole-1-yloxytris(dimethylamino)phosphonium hexafluorophosphate. The reaction proceeds rapidly under mild conditions in tetrahydrofuran with the addition of N,N-diisopropylethylamine to form alcohols in high yield (McGeary R. P.: Tetrahedron Letters, 39, 3319-3322, 1998).

Carboxyl groups of tetrasaccharide esterified to methyl esters, which also contain unprotected hydroxyl groups, can be reduced by NaBH₄ in methanol. Reduction to relevant alcohol proceeds for 1 hour at 20° C. (D'Acquarica I. et al.: Tetrahedron, 58, 51, 10127-10136, 2002).

Aliphatic carboxylic acids containing amide and pyrazole groups can be reduced after activation with 1,1′-carbonyldiimidazole using NaBH₄ in tetrahydrofuran, water system. The reduction proceeds at 0° C. in 30 minutes (EP 2511265, 2012).

A similar approach was described in the article (Stolz F. et al.: European Journal of Organic Chemistry, 15, 3304-3312, 2004) where aliphatic carboxylic acids containing also ether groups were reduced by NaBH₄ after activation of carboxyl with 1,1′-carbonyldiimidazole. The reaction proceeds in ethanol, dichloromethane, N,N-dimethylformamide system.

Reductions of carboxylic acids, which also contain alkynes, ethers and N-heterocycles, were described in patent publication (CN112778310, 2021). The carboxyl was activated to form methyl ester and the subsequent reduction proceeds with NaBD₄ in tetrahydrofuran after 2 hours at 20° C.

A disaccharide with free hydroxyl groups and cholesterol linked to the reducing end by 0-glycoside bond was reduced at carboxyl following its esterification with diazomethane in methanol. NaBH₄ was used as reagent and the reaction proceeds for 2 hours in methanol at ambient temperature (Yoshikawa K. et al.: Chemical and Pharmaceutical Bulletin, 46, 7, 1102-1107, 1998).

Ethyl chloroformate as an activating agent for aliphatic carboxylic acids containing epoxides in the molecule as well was also used in patent publication WO 056548, 2005. The activation itself proceeds in dichloromethane and the subsequent reduction with NaBH₄ proceeds at 0-20° C. with the addition of ethanol.

A similar process is described in another patent WO 123133, 2015, where aliphatic carboxylic acids containing pyrazole in the molecule were activated by ethyl chloroformate in tetrahydrofuran and the subsequent reduction with NaBH₄ at 20° C. was also proceeding with the addition of ethanol.

In addition to ethyl chloroformate, its analogues such as methyl chloroformate (CN 104672288, 2017) or isobutylchloroformate (EP 3239143, 2017) can be used to activate carboxylic acids. In both cases, the activation can be achieved in tetrahydrofuran and the subsequent reduction with NaBH₄ proceeds in tetrahydrofuran or in its mixture with water.

Activation of carboxylic acids where oxazole is present in the molecule can be achieved by SOCl₂ in dichloromethane. Subsequent reduction with LiB(Et)₃H proceeds in tetrahydrofuran at boiling point (Kanemasa S., Onimura K., Tetrahedron, 48, 40, 8645-8658, 1992).

Activation of carboxylic acids on monosaccharides, where all hydroxyl groups are protected as acetyls, can be achieved by PCl₅ in diethyl ether (Soroka N. V. et al.: Carbohydrate Research, 340, 4, 539-546, 2005). The subsequent rapid reduction with NaBH₄ proceeds in a tetrahydrofuran/water mixture at 0° C.

If unprotected hydroxyl groups are present, carboxyl reduction can be achieved after activation with acetylchloride. The subsequent reduction with NaBH₄ proceeds in ethanol at lower temperature (Lundt I. et al: Tetrahedron, 50, 46, 13285-13298, 1994), (Lundt I. et al: Synthesis, 7, 714-720, 1993).

In general, when reducing carboxylic acids with boron-based hydrides, it is necessary to activate the carboxyl to its more reactive (electrophilic) derivative. The activation usually proceeds under aprotic conditions and the subsequent reduction with NaBH₄ usually proceeds in mixtures with protic solvents such as methanol, ethanol or water. Compounds containing hydroxyl and amide groups were also used as reduced substrates.

Reduction after Activation of Carboxyl Groups with Halogenated 1,3,5-Triazines:

Activation of aliphatic carboxylic acids and N-protected amino acids can be achieved together with reduction by means of NaBH₄ in one reaction step and without solvent. A mixture of 2,4,6-trichloro-1,3,5-triazine and triphenyl phosphine was used as the activator (Subin J. et al: RSC Adv., 4, 46947-46950, 2014).

A similar approach was described in the article where 2,4,6-trifluoro-1,3,5-triazine in dichloromethane was used at temperatures of −20 to −10° C. for activation of a carboxylic acid containing a lactone, followed by a rapid reduction using NaBH₄ at 20° C. (Kokotos G. et al.: Journal of Organic Chemistry, 61, 20, 6994-6996, 1996).

Halogenated 1,3,5-triazines are very reactive and therefore activation of carboxyl compounds proceeds in aprotic systems or without solvent.

### Enzymatic Reduction

Aliphatic carboxylic acids containing ethers and acetamides can be reduced in water by the enzyme Gloeosporium olivarum at 27° C. However, the reaction time of 768 hours is extremely long, and racemization of the alpha position of the reduced carbonyl has also been observed (Tsuda Y. et al.: Chemical and Pharmaceutical Bulletin, 33, 5, 1955-1960, 1985).

Another enzyme capable of reducing aliphatic carboxylic acids containing ether groups is Glomerella cingulata. This enzyme works at 27° C. in water, the reaction time is up to 624 hours, and racemization of the alpha position of carbonyl has also been observed (Tsuda Y. et al.: Agricultural and Biological Chemistry, 48, 5, 1373-1374, 1984.

When using the enzyme Glomerella cingulata with the addition of KH₂PO₄, MgSO₄, peptone and sucrose, the reaction time is only 24 hours in water at 27° C. (Tsuda Y.: Chemical and pharmaceutical bulletin, 35, 6, 2554-2557, 1987).

In general, reductions can be made even in mild conditions in water using an enzyme, but it is necessary to expect a longer reaction time, as well as the racemization of the alpha position of the carbonyl.

Reduction of Polysaccharides after Activation with Carbodiimines

Activation of polysaccharides carboxyl group (heparin, chondroitin sulfate) with a large excess of 1-ethyl-3-(3-dimethylaminopropyl)carbodiimide and a subsequent reduction with a large excess of NaBH₄ was described in the article (Inoue Y. et al.: Carbohydrate Research, 111, 113-125, 1982). Reactions proceed in water and hyaluronic acid was not mentioned among the reduced substrates.

Hyaluronic acid reduced on carboxyl with a degree of modification of 20% (ratio of reduced to non-reduced disaccharides is 1/4) was described as a product in the analytical confirmation of the presence of O-acyl isourea as a by-product of the preparation of amides on hyaluronic acid carboxyl by activation using 1-ethyl-3-(3-dimethylaminopropyl) carbodiimide (Ponedelkina I. Y. et al.: Russian Journal of Bioorganic Chemistry, 31, 1, 82-86, 2005). The O-acylisourea reduction was performed using an extreme excess of NaBH₄ (40-times the saccharide weight). The reaction proceeds at room temperature for 2.5 hours and the pH is maintained at 7 to 8 by the addition of 4M HCl solution. Neither racemization at the alpha carboxyl position nor the effect of reaction conditions on hyaluronic acid molecular weight degradation were investigated (molecular weight values were not given even for the input material).

In the patent publication (US0203056, 2005) hyaluronic acid is claimed, where the —OH groups are sulfated to the group —OSO₃Na in different positions with a high degree of modification and at the same time all carboxyl groups are reduced to primary alcohol (degree of modification 100%). The structure of the claimed material includes racemization at position 5 of the polysaccharide cycle (the alpha position of the original carboxylic acid) and at the same time the presence of the unreduced anomeric end of the polysaccharide. A large excess of 1-ethyl-3-(3-dimethylaminopropyl) carbodiimide as an activator (5 to 20 eq) and a large excess of NaBH₄ (10 to 300 eq) were used in the preparation process, the reaction proceeds for 2 hours at 50° C. and pH in the range from 4 to 5.

Use of Acetonitrile for Carboxyl Groups Activation or Reduction with NaBH₄.

In the article (Petukhov P. A. et al.: Tetrahedron, 53, 7, 2535-2550, 1997), the reduction of oximes with NaBH₄ in acetonitrile is described, where the resulting intermediate R═N—O—BH₃Na reacts with acetonitrile so that two or three hydrogens (bound to a boron atom) are replaced by the group —N═CH—CH₃. So, there is an addition of some forms of borohydrides to the triple bond of acetonitrile. The authors even observed and demonstrated the formation of the ethylamine-BH₃ complex at a higher temperature using NMR.

The use of acetonitrile as a co-solvent in the synthesis of hyaluronic acid benzylamides by activation with a 1,3,5-triazine-based reagent (4-(4,6-dimethoxy-1,3,5-triazine-2-yl)-4-methylmorpholinium chloride) was studied in detail in the diploma thesis (Rydergren S.: Chemical modification of Hyaluronan using DMTMM-activated amidation, 2013). It was observed that the increasing presence of acetonitrile (0 to 30 vol. %) in aqueous phosphate buffer dramatically reduces the degree of modification to the final benzylamide (at 0 vol. % of acetonitrile, 35% degree of modification was achieved and at 30 vol. % of acetonitrile, only 5% of the degree of modification was achieved). The formation of amides is conditioned by the formation of the same reactive intermediate (Scheme 1, ester) as the formation of the primary alcohol in the process claimed in this invention (Scheme 1).

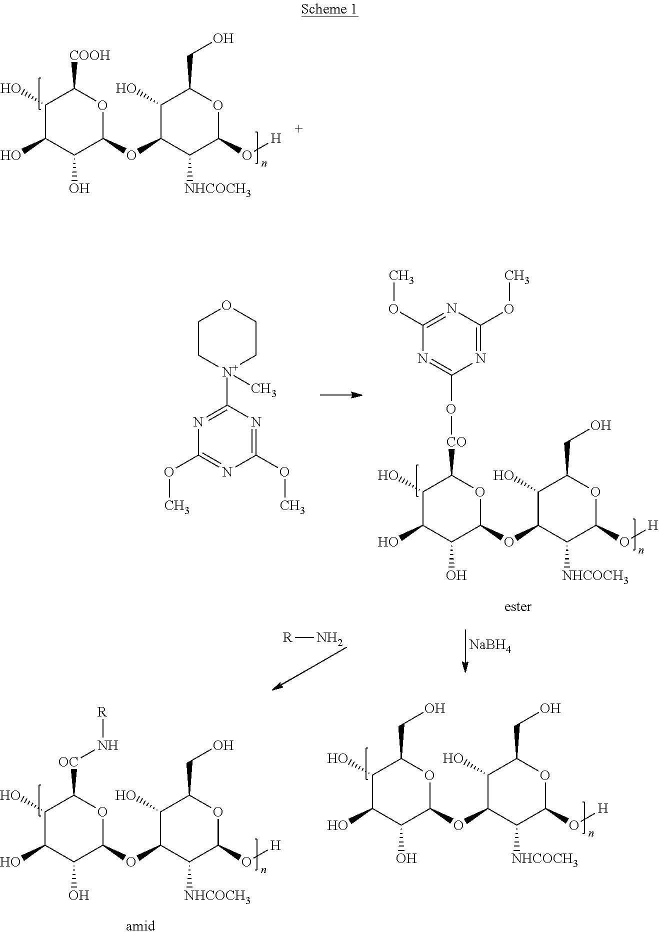

In general, polysaccharides based on hyaluronic acid according to the structural formula I have not yet been described. Similar polysaccharides with a lower degree of modification or a significantly lower molecular weight due to the harsher conditions used in the modification (Examples 1 to 4) were mentioned. The successful method of preparation using an activator (4-(4,6-dimethoxy-1,3,5-triazine-2-yl)-4-methylmorpholinium chloride in combination with NaBH₄ in water and acetonitrile is surprising because of the possible reaction of acetonitrile with NaBH₄ (Petukhov P. A. et al.: Tetrahedron, 53, 7, 2535-2550, 1997) and also because of the suppression of the formation of a reactive intermediate—ester (Rydergren S.: Chemical modification of Hyaluronan using DMTMM-activated amidation, 2013) necessary for a successful reduction. The surprisingness of the use of acetonitrile is enhanced by the examples given in this invention, which describe that the presence of other co-solvents either significantly reduces the degree of modification, such as DMF (Example 7), tert-butanol (Example 8), glycerol (Example 9), ethylene glycol (Example 10) or no co-solvent to water (Example 6), or reduces the molecular weight of the final polymer, such as DMSO (Example 15), formamide (Example 11), methanol (Example 12), dioxane (Example 13).

The present invention solves the preparation and use of a hyaluronic acid-based polysaccharide having a reduced polarity caused by the exchange of more polar carboxyl groups for less polar primary alcohols. At the same time, it can be expected that replacing glucuronic acid with glucose in the polymer structure without other side-reactions (configuration inversion, reactions on hydroxyl or amide groups) allows maintaining a high biocompatibility and biodegradability. The possibility to prepare materials with molecular weights up to 1,000 kg·mol⁻¹ allows this material to be applied at areas where a longer biocompatible polymer with an increased resistance to enzymatic and/or thermal cleavage or a reduced swelling is required.

### SUMMARY OF THE INVENTION

The subject of the invention is a derivative of hyaluronic acid or its salt, which has the form of a linear chain and in which some carboxyl groups are replaced by the group —CH₂—OH according to the structural formula Z,

I═R¹-(A)_{X}-(B)_{Y}—R²
wherein A is a structural fragment of a linear chain:

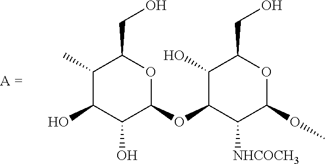

B is a structural fragment of a linear chain:

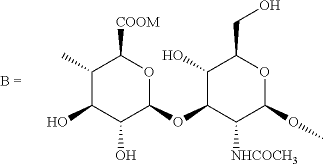

R¹ is the structural fragment of the end of the linear chain:

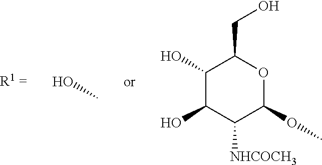

R² is the structural fragment of the end of a linear chain:

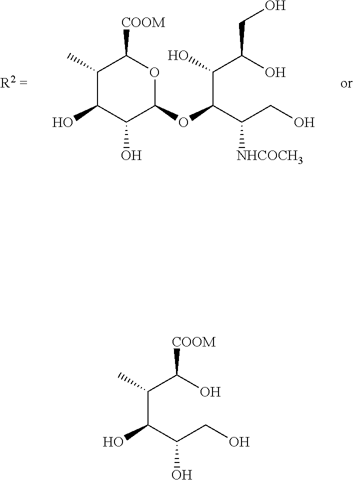

M can be hydrogen or any alkali metal cation,
X is an index indicating the number of structural fragments A within the chain,
Y is an index indicating the number of structural fragments B within the chain, while at the same time:
   structural fragments A and B are distributed in the linear chain randomly, and
   the sum of the X+Y indexes is in the range of 500 to 2,500, and
   the ratio of the indexes X/Y is in the range of 3/1 to 1/4.

The derivative according to the invention can be used for viscosupplementation, anti-wrinkle dermal fillers, fascia fillers, eye drops, or for preparing implantable medical devices.

Furthermore, the invention relates to the method of production of the polysaccharide according to the structural formula I, where the modification of hyaluronic acid is carried out using an activator 4-(4,6-dimethoxy-1,3,5-triazine-2-yl)-4-methylmorpholinium chloride (DMTMM) and sodium tetrahydride borate (NaBH₄) in a mixture of water and acetonitrile, the presence of acetonitrile playing an important role in the possibility of achieving a higher molecular weight and at the same time a more significant degree of modification. Specifically, the method of production consists in preparing an aqueous solution of hyaluronic acid having a molecular weight in the range of 400 to 2,200 kg·mol⁻¹, preferably 800 to 1,400 kg·mol⁻¹, the concentration thereof being in the range of 0.3 to 2 wt. %, preferably 0.7 to 1.3 wt. %, adding acetonitrile in an amount of 50-100%, preferably 75-90% of the volume of the aqueous solution, then adding 4-(4,6-dimethoxy-1,3,5-triazine-2-yl)-4-methylmorpholinium chloride in an amount of 0.7 to 2.3, preferably 0.9 to 1.2 wt. equivalent in relation to hyaluronic acid, and allowing the mixture to react for 15 to 30 hours, preferably 20 to 25 hours, at 15 to 25° C. Then 0.5 to 2.3, preferably 0.7 to 1.1 wt. equivalent of NaBH₄, is added and the solution is stirred for 2 to 30 hours, preferably 4 to 8 hours at 15 to 25° C. Subsequently, acetic acid is gradually added to the solution until a pH of 4.5 to 5.5 is reached and the mixture is stirred for 1.5 to 3 hours at 15 to 25° C. The final reaction mixture is then isolated, for example by precipitation, e.g., using isopropanol, ethanol, acetone or acetonitrile, preferably isopropanol, or chromatographically, to produce the desired reduced product in a powder form.

The process described in this invention is technologically more preferable than analogous processes reducing carboxyl groups of polymers mainly from the point of view of using a significantly lower amount of activator and reducing agent (one equivalent versus tens to hundreds of equivalents). The examples given in the present invention (Examples 18 to 24) also showed that surprisingly only the combination of the activating agent DMTMM and acetonitrile as co-solvent achieved more significant degrees of modification (the ratio of the indexes X/Y is more than 1/4, DS more than 20%, preferably X/Y is at least 1/3, i.e., DS at least 25%) and at the same time significantly higher molecular weight values (500 to 1,000 kg·mol-¹). The application of the DMTMM activating agent with other co-solvents, or the application of a standard EDC activating agent with different co-solvents (including acetonitrile) or without co-solvents, results in materials with a molecular weight of 20 to 200 kg·mol-1 (Examples 7 to 17). The molecular weight of the resulting product according to the invention is more than 200 kg/mol and can reach up to 1,000 kg/mol, preferably is in the range of 300 to 1,000 kg/mol, the most preferably 500 to 1,000 kg/mol.

The procedure described above can be repeated 2 to 5 times on the final product to obtain a higher degree of modification while maintaining a high molecular weight.

The subject of the invention are also compositions containing a polysaccharide according to the structural formula I, which are characterized by an increased resistance to a decrease of molecular weight under the action of heat or enzymes degrading hyaluronic acid. The differences in resistance compared to the native hyaluronic acid are described in Examples 45 and 46 (heat resistance) and in Example 47 (enzymatic resistance). The composition according to the invention includes a derivative of hyaluronic acid according to the structural formula I in a concentration of 0.01 to 99% and an additive selected from the group comprising water, sodium chloride, calcium chloride, glycerol, hyaluronic acid, chondroitin sulfate. In one embodiment, the composition preferably has the form of a solution or gel in an aqueous solvent. An aqueous solvent means, for example, pure water or water containing any chemically non-reactive additives such as drugs, vitamins, growth factors, ethanol, glycerol, etc. In another embodiment, the composition has the form of a solid substrate, for example, a lyophilizate or a powder.

Furthermore, the invention relates to the use of polysaccharide according to the structural formula I in areas where the following characteristics are required, or a combination thereof:
use of biocompatible and biodegradable polymer
increased resistance to thermal degradation compared to the native hyaluronic acid
increased resistance to enzymatic degradation compared to the native hyaluronic acid
possibility to regulate the rate of degradation
increased viscosity of solutions
reduced swelling of solution or solid forms.

Due to the increased resistance, the final composition can be preferably used mainly in applications such as viscosupplementation, anti-wrinkle dermal fillers, solutions for fascia fillers, eye drops, solutions for storing of contact lenses, or for preparing implantable medical devices.

This research was supported by the European Regional Development Fund—INBIO project (reg. no.: CZ.02.1.01/0.0/0.0/16_026/0008451).

### DESCRIPTION OF THE DRAWINGS

FIG. 1—Effect of materials prepared according to Examples 20 and 23 on the viability of 3T3 fibroblasts

FIG. 2—Effect of materials prepared according to Examples 23 and 28 on the viability of HaCaT keratinocytes

FIG. 3—Effect of materials prepared according to Examples 21 and 28 on the viability of HaCaT keratinocytes after drying

FIG. 4—Comparison of the degradation rate of 0.5% solutions of materials prepared according to Examples 20, 22 and 28 (solid line) and native hyaluronic acid (dashed line) after heating to 80° C. in phosphate buffer (pH 7.4)

FIG. 5—Comparison of the degradation rate of the material prepared according to Example 39 (solid line) and native hyaluronic acid (dashed line) after heating to 120° C. in the form of a powder

FIG. 6—Comparison of the enzymatic degradation rate of solutions of materials prepared according to Examples 22 and 28 (solid lines) and the native hyaluronic acid (dashed lines) after application of the bovine testicular hyaluronidase (BTH) enzyme.

FIG. 7—In vivo biocompatibility and safety in subcutaneous application of materials prepared according to Examples 21 and 37.

### EXAMPLES OF EMBODIMENTS

DS=degree of substitution=100%*(molar amount of modified polysaccharide unit)/(molar amount of all polysaccharide units)
eq.=molar equivalent per molar amount of disaccharide
DMTMM=4-(4,6-dimethoxy-1,3,5-triazine-2-yl)-4-methylmorpholinium chloride
EDC=1-ethyl-3-(3-dimethylaminopropyl) carbodiimide
HA=hyaluronic acid
Mw=molecular weight
NMR analysis (500 MHz, D₂O/NaOD, δ ppm): 3.16 (1H; glucose cycle, position 2)
BTH bovine testicular hyaluronidase

The term equivalent (eq) used herein refers unless otherwise indicated to a repeating unit of the polysaccharide in question, such as hyaluronic acid disaccharide. Percentages are given as weight percentages, unless otherwise indicated. The molecular weight of the starting polysaccharides is the weight average molecular weight and is determined by the SECMALLS method.

The hyaluronic acid salt refers to a salt, which is formed from hyaluronic acid after replacement of the carboxyl group protons with other cations, such as alkali metal cations.

### Example 1 (Comparative Example)

### Hyaluronic Acid Reduction Using EDC and NaBH₄ in Water

To a solution of HA (100 mg, 1,350 kg·mol⁻¹) in 15 mL of water, HCl was added until pH 4.7. Then EDC (448 mg) was added, keeping the pH at 4.7 with the gradual addition of HCl. The mixture was then stirred for 24 hours at 20° C. Then ten times NaBH₄ solution (0.179 g in 2.5 mL of water, totally 25 mL of solution) was added and the suspension was stirred for further 2 hours at 50° C. Subsequently, the HCl was added to the solution until pH 7.0 and the mixture was stirred for 2 hours at 20° C. The resulting solution was then dialysed and subsequently evaporated at reduced pressure, the solid was filtered out and dried at reduced pressure. The product was analysed using NMR, DS 85% and SECMALLS, Mw 32 kg·mol⁻¹.

### Example 2 (Comparative Example)

### Hyaluronic Acid Reduction Using EDC and NaBH₄ in Water

To a solution of HA (100 mg, 1,800 kg·mol⁻¹) in 10 mL of water, EDC was added (100 mg), keeping the pH at 4.5 with the gradual addition of HCl. The mixture was then stirred for 2 hours at 20° C. Subsequently, NaBH₄ (1 g) was added (300 mg) and the suspension was stirred for further 2 hours at 20° C. The resulting solution was then dialysed and subsequently evaporated at reduced pressure, the solid was filtered out and dried at reduced pressure. The product was analysed using NMR, DS 77% and SECMALLS, Mw 137 kg·mol⁻¹.

### Example 3 (Comparative Example)

### Hyaluronic Acid Reduction Using EDC and NaBH₄ in Water

To a solution of HA (100 mg, 1,800 kg·mol⁻¹) in 10 mL of water, EDC was added (300 mg), keeping the pH at 4.5 with the gradual addition of HCl. The mixture was then stirred for 2 hours at 20° C. Subsequently, NaBH₄ (300 mg) was added and the suspension was stirred for further 2 hours at 20° C. The resulting solution was then dialysed and subsequently evaporated at reduced pressure, the solid was filtered out and dried at reduced pressure. The product was analysed using NMR, DS 77% and SECMALLS, Mw 63 kg·mol⁻¹.

### Example 4 (Comparative Example)

### Hyaluronic Acid Reduction Using EDC and NaBH₄ in Water and Acetonitrile

To a solution of HA (100 mg, 1,800 kg·mol⁻¹) in 10 mL of water and 8.6 mL of acetonitrile, EDC was added (300 mg), keeping the pH at 4.5 by means of a gradual addition of HCl. The mixture was then stirred for 2 hours at 20° C. Subsequently, NaBH₄ (100 mg) was added and the suspension was stirred for further 2 hours at 20° C. The resulting solution was then dialysed and subsequently evaporated at reduced pressure, the solid was filtered out and dried at reduced pressure. The product was analysed using NMR, DS 77% and SECMALLS, Mw 44 kg·mol⁻¹.

### Example 5 (Comparative Example)

### Hyaluronic Acid Reduction Using DMTMM and NaBH₄ in Water

To a solution of HA (10 mg, 180 kg·mol⁻¹) in 0.7 mL of water, DMTMM (10 mg) was added and the mixture was stirred for 24 hours at 20° C. Then NaBH₄ (10 mg) was added and the solution was stirred for further 20 hours at 20° C. Subsequently, CH₃COOH was added to the solution until pH 4.9 and the mixture was stirred for 2 hours at 20° C. The resulting solution was then precipitated by 3 mL of isopropanol, the solid was filtered out and dried at reduced pressure. The product was analysed using NMR, DS 20%.

### Example 6 (Comparative Example)

### Hyaluronic Acid Reduction Using DMTMM and NaBH₄ in Water

To a solution of HA (10 mg, 1,350 kg·mol⁻¹) in 0.7 mL of water, DMTMM (10 mg) was added and the mixture was stirred for 24 hours at 20° C. Then NaBH₄ (10 mg) was added and the solution was stirred for further 20 hours at 20° C. Subsequently, CH₃COOH was added to the solution until pH 5.0 and the mixture was stirred for 2 hours at 20° C. The resulting solution was then precipitated by 3 mL of isopropanol, the solid was filtered out and dried at reduced pressure. The product was analysed using NMR, DS 9%.

### Example 7 (Comparative Example)

### Hyaluronic Acid Reduction Using DMTMM and NaBH₄ in Water and DMF

To a solution of HA (10 mg, 1,350 kg·mol⁻¹) in 1 mL of water and 0.86 mL of DMF, DMTMM (10 mg) was added and the mixture was stirred for 24 hours at 20° C. Then NaBH₄ (10 mg) was added and the solution was stirred for further 20 hours at 20° C. Subsequently, CH₃COOH was added to the solution until pH 5.0 and the mixture was stirred for 2 hours at 20° C. The resulting solution was then precipitated by 3 mL of isopropanol, the solid was filtered out and dried at reduced pressure. The product was analysed using NMR, DS 22%.

### Example 8 (Comparative Example)

### Hyaluronic Acid Reduction Using DMTMM and NaBH₄ in Water and Tert-Butanol

To a solution of HA (10 mg, 1,350 kg·mol⁻¹) in 1 mL of water and 0.86 mL of tert-butanol, DMTMM (10 mg) was added and the mixture was stirred for 24 hours at 20° C. Then NaBH₄ (10 mg) was added and the solution was stirred for further 20 hours at 20° C. Subsequently, CH₃COOH was added to the solution until pH 5.1 and the mixture was stirred for 2 hours at 20° C. The resulting solution was then precipitated by 3 mL of isopropanol, the solid was filtered out and dried at reduced pressure. The product was analysed using NMR, DS 15%.

### Example 9 (Comparative Example)

### Hyaluronic Acid Reduction Using DMTMM and NaBH₄ in Water and Glycerol

To a solution of HA (10 mg, 1,350 kg·mol⁻¹) in 1 mL of water and 0.86 mL of glycerol, DMTMM (10 mg) was added and the mixture was stirred for 24 hours at 20° C. Then NaBH₄ (10 mg) was added and the solution was stirred for a further 20 hours at 20° C. Subsequently, CH₃COOH was added to the solution until pH 4.5 and the mixture was stirred for 2 hours at 20° C. The resulting solution was then precipitated by 3 mL of isopropanol, the solid was filtered out and dried at reduced pressure. The product was analysed using NMR, DS 7%.

### Example 10 (Comparative Example)

### Hyaluronic Acid Reduction Using DMTMM and NaBH₄ in Water and Ethylene Glycol

To a solution of HA (10 mg, 1,350 kg·mol⁻¹) in 1 mL of water and 0.86 mL of ethylene glycol, DMTMM (10 mg) was added and the mixture was stirred for 24 hours at 20° C. Then NaBH₄ (10 mg) was added and the solution was stirred for further 20 hours at 20° C. Subsequently, CH₃COOH was added to the solution until pH 4.7 and the mixture was stirred for 2 hours at 20° C. The resulting solution was then precipitated by 3 mL of isopropanol, the solid was filtered out and dried at reduced pressure. The product was analysed using NMR, DS 14%.

### Example 11 (Comparative Example)

### Hyaluronic Acid Reduction Using DMTMM and NaBH₄ in Formamide

To a solution of HA (10 mg, 1,350 kg·mol⁻¹) in 1 mL of formamide, DMTMM (10 mg) was added and the mixture was stirred for 24 hours at 20° C. Then NaBH₄ (10 mg) was added and the solution was stirred for further 20 hours at 20° C. Subsequently, 0.02 mL of CH₃COOH was added to the solution and the mixture was stirred for 2 hours at 20° C. The resulting solution was then precipitated by 3 mL of isopropanol, the solid was filtered out and dried at reduced pressure. The product was analysed using NMR, DS 43% and Mw 142 kg·mol⁻¹.

### Example 12 (Comparative Example)

### Hyaluronic Acid Reduction Using DMTMM and NaBH₄ in Water and Methanol

To a solution of HA (10 mg, 1,350 kg·mol⁻¹) in 1 mL of water and 0.86 mL of methanol, DMTMM (10 mg) was added and the mixture was stirred for 24 hours at 20° C. Then NaBH₄ (10 mg) was added and the solution was stirred for further 20 hours at 20° C. Subsequently, CH₃COOH was added to the solution until pH 5.0 and the mixture was stirred for 2 hours at 20° C. The resulting solution was then precipitated by 3 mL of isopropanol, the solid was filtered out and dried at reduced pressure. The product was analysed using NMR, DS 39% and Mw 126 kg·mol⁻¹.

### Example 13 (Comparative Example)

### Hyaluronic Acid Reduction Using DMTMM and NaBH₄ in Water and Dioxane

To a solution of HA (10 mg, 1,350 kg·mol⁻¹) in 1 mL of water and 0.86 mL of dioxane, DMTMM (10 mg) was added and the mixture was stirred for 24 hours at 20° C. Then NaBH₄ (10 mg) was added and the solution was stirred for further 20 hours at 20° C. Subsequently, CH₃COOH was added to the solution until pH 5.3 and the mixture was stirred for 2 hours at 20° C. The resulting solution was then precipitated by 3 mL of isopropanol, the solid was filtered out and dried at reduced pressure. The product was analysed using NMR, DS 33% and Mw 196 kg·mol⁻¹.

### Example 14 (Comparative Example)

### Hyaluronic Acid Reduction Using DMTMM and NaBH₄ in Water and Tetrahydrofuran

To a solution of HA (10 mg, 1,350 kg·mol⁻¹) in 1 mL of water and 0.86 mL of tetrahydrofuran, DMTMM (10 mg) was added and the mixture was stirred for 24 hours at 20° C. Then NaBH₄ (10 mg) was added and the solution was stirred for further 20 hours at 20° C. Subsequently, CH₃COOH was added to the solution until pH 5.1 and the mixture was stirred for 2 hours at 20° C. The resulting solution was then precipitated by 3 mL of isopropanol, the solid was filtered out and dried at reduced pressure. The product was analysed using NMR, DS 24%.

### Example 15 (Comparative Example)

### Hyaluronic Acid Reduction Using DMTMM and NaBH₄ in Water and DMSO

To a solution of HA (1 g, 1,350 kg·mol⁻¹) in 140 mL of water and 100 mL of DMSO, DMTMM (2 g) was added and the mixture was stirred for 24 hours at 20° C. Then NaBH₄ (2 g) was added and the solution was stirred for further 20 hours at 20° C. Subsequently, CH₃COOH was added to the solution until pH 5.4 and the mixture was stirred for 2 hours at 20° C. The resulting solution was then precipitated by 350 mL of isopropanol, the solid was filtered out and dried at reduced pressure. The product was analysed using NMR, DS 43% and SECMALLS, Mw 186 kg·mol⁻¹.

### Example 16 (Comparative Example)

### Hyaluronic Acid Reduction Using DMTMM and NaBH₄ in Water and DMSO

To a solution of reduced HA prepared according to preceding example (500 mg, 186 kg·mol⁻¹) in 70 mL of water and 50 mL of DMSO, DMTMM (1 g) was added and the mixture was stirred for 24 hours at 20° C. Then NaBH₄ (1 g) was added and the solution was stirred for further 20 hours at 20° C. Subsequently, CH₃COOH was added to the solution until pH 5.4 and the mixture was stirred for 2 hours at 20° C. The resulting solution was then precipitated by 250 mL of isopropanol, the solid was filtered out and dried at reduced pressure. The product was analysed using NMR, DS 61% and SECMALLS, Mw 128 kg·mol⁻¹.

### Example 17 (Comparative Example)

### Hyaluronic Acid Reduction Using DMTMM and NaBH₄ in Water and DMSO

To a solution of HA (500 mg, 1,350 kg·mol⁻¹) in 70 mL of water and 50 mL of DMSO, DMTMM (1 g) was added and the mixture was stirred for 24 hours at 20° C. Then NaBH₄ (1 g) was added and the solution was stirred for further 20 hours at 20° C. Subsequently, CH₃COOH was added to the solution until pH 5.2 and the mixture was stirred for 2 hours at 20° C. The resulting solution was then precipitated by 250 mL of isopropanol, the solid was filtered out and dried at reduced pressure. The product was analysed using NMR, DS 42% and SECMALLS, Mw 198 kg·mol⁻¹.

### Example 18

### Hyaluronic Acid Reduction Using DMTMM and NaBH₄ in Water and Acetonitrile

To a solution of HA (100 mg, 1,350 kg·mol⁻¹) in 14 mL of water and 8 mL of acetonitrile, DMTMM (100 mg) was added and the mixture was stirred for 24 hours at 20° C. Then NaBH₄ (100 mg) was added and the solution was stirred for further 20 hours at 20° C. Subsequently, CH₃COOH was added to the solution until pH 5.1 and the mixture was stirred for 2 hours at 20° C. The resulting solution was then precipitated by 35 mL of isopropanol, the solid was filtered out and dried at reduced pressure. The product was analysed using NMR, DS 25% and SECMALLS, Mw 922 kg·mol⁻¹.

### Example 19

### Hyaluronic Acid Reduction Using DMTMM and NaBH₄ in Water and Acetonitrile

To a solution of HA (1 g, 1,350 kg·mol⁻¹) in 100 mL of water and 86 mL of acetonitrile, DMTMM (1 g) was added and the mixture was stirred for 24 hours at 20° C. Then NaBH₄ (1 g) was added and the solution was stirred for further 20 hours at 20° C. Subsequently, CH₃COOH was added to the solution until pH 5.5 and the mixture was stirred for 2 hours at 20° C. The resulting solution was then precipitated by 350 mL of isopropanol, the solid was filtered out and dried at reduced pressure. The product was analysed using NMR, DS 30% and SECMALLS, Mw 872 kg·mol⁻¹.

### Example 20

### Reduction of Reduced Hyaluronic Acid Using DMTMM and NaBH₄ in Water and Acetonitrile

To a solution of reduced HA prepared according to preceding example (500 mg, 872 kg·mol⁻¹) in 50 mL of water and 43 mL of acetonitrile, DMTMM (500 mg) was added and the mixture was stirred for 24 hours at 20° C. Then NaBH₄ (500 mg) was added and the solution was stirred for further 20 hours at 20° C. Subsequently, CH₃COOH was added to the solution until pH 5.5 and the mixture was stirred for 2 hours at 20° C. The resulting solution was then precipitated by 35 mL of isopropanol, the solid was filtered out and dried at reduced pressure. The product was analysed using NMR, DS 50% and SECMALLS, Mw 690 kg·mol⁻¹.

### Example 21

### Hyaluronic Acid Reduction Using DMTMM and NaBH₄ in Water and Acetonitrile

To a solution of HA (1 g, 1,350 kg·mol⁻¹) in 100 mL of water and 86 mL of acetonitrile, DMTMM (1 g) was added and the mixture was stirred for 15 hours at 25° C. Then NaBH₄ (1 g) was added and the solution was stirred for further 15 hours at 25° C. Subsequently, CH₃COOH was added to the solution until pH 4.5 and the mixture was stirred for 2 hours at 20° C. The resulting solution was then precipitated by 350 mL of isopropanol, the solid was filtered out and dried at reduced pressure. The product was analysed using NMR, DS 29% and SECMALLS, Mw 823 kg·mol⁻¹.

### Example 22

### Hyaluronic Acid Reduction Using DMTMM and NaBH₄ in Water and Acetonitrile

To a solution of HA (500 mg, 1,350 kg·mol⁻¹) in 50 mL of water and 43 mL of acetonitrile, DMTMM (500 mg) was added and the mixture was stirred for 24 hours at 20° C. Then NaBH₄ (500 mg) was added and the solution was stirred for further 30 hours at 15° C. Subsequently, CH₃COOH was added to the solution until pH 4.8 and the mixture was stirred for 3 hours at 15° C. The resulting solution was then precipitated by 250 mL of isopropanol, the solid was filtered out and dried at reduced pressure. The product was analysed using NMR, DS 31% and SECMALLS, Mw 886 kg·mol⁻¹.

### Example 23

### Hyaluronic Acid Reduction Using DMTMM and NaBH₄ in Water and Acetonitrile

To a solution of HA (10 g, 1,350 kg·mol⁻¹) in 1,000 mL of water and 860 mL of acetonitrile, DMTMM (10 g) was added and the mixture was stirred for 24 hours at 20° C. Then NaBH₄ (10 g) was added and the solution was stirred for further 20 hours at 20° C. Subsequently, CH₃COOH was added to the solution until pH 4.9 and the mixture was stirred for 1.5 hours at 25° C. The resulting solution was then precipitated by 3,000 mL of isopropanol, the solid was filtered out and dried at reduced pressure. The product was analysed using NMR, DS 29% and SECMALLS, Mw 850 kg·mol⁻¹.

### Example 24

### Hyaluronic Acid Reduction Using DMTMM and NaBH₄ in Water and Acetonitrile

To a solution of HA (10 g, 1,350 kg·mol⁻¹) in 1,000 mL of water and 860 mL of acetonitrile, DMTMM (10 g) was added and the mixture was stirred for 30 hours at 15° C. Then NaBH₄ (10 g) was added and the solution was stirred for further 30 hours at 15° C. Subsequently, CH₃COOH was added to the solution until pH 4.9 and the mixture was stirred for 2 hours at 20° C. The resulting solution was then precipitated by 250 mL of isopropanol, the solid was filtered out and dried at reduced pressure. The product was analysed using NMR, DS 30% and Mw 888 kg·mol⁻¹.

### Example 25

### Hyaluronic Acid Reduction Using DMTMM and NaBH₄ in Water and Acetonitrile

To a solution of HA (10 g, 1,800 kg·mol⁻¹) in 1,000 mL of water and 860 mL of acetonitrile, DMTMM (10 g) was added and the mixture was stirred for 24 hours at 20° C. Then NaBH₄ (10 g) was added and the solution was stirred for further 20 hours at 20° C. Subsequently, CH₃COOH was added to the solution until pH 5.1 and the mixture was stirred for 2 hours at 20° C. The resulting solution was then precipitated by 3,000 mL of isopropanol, the solid was filtered out and dried at reduced pressure. The product was analysed using NMR, DS 26% and Mw 877 kg·mol⁻¹.

### Example 26

### Reduction of Reduced Hyaluronic Acid Using DMTMM and NaBH₄ in Water and Acetonitrile

To a solution of reduced HA prepared according Example 25 (9.6 g) in 1,000 mL of water and 860 mL of acetonitrile, DMTMM (10 g) was added and the mixture was stirred for 24 hours at 20° C. Then NaBH₄ (10 g) was added and the solution was stirred for further 20 hours at 20° C. Subsequently, CH₃COOH was added to the solution until pH 4.9 and the mixture was stirred for 2 hours at 20° C. The resulting solution was then precipitated by 3,000 mL of isopropanol, the solid was filtered out and dried at reduced pressure. The product was analysed using NMR, DS 43% and Mw 729 kg·mol⁻¹.

### Example 27

### Reduction of Reduced Hyaluronic Acid Using DMTMM and NaBH₄ in Water and Acetonitrile

To a solution of reduced HA (9.2 g) prepared according Example 26 in 1,000 mL of water and 860 mL of acetonitrile, DMTMM (10 g) was added and the mixture was stirred for 24 hours at 20° C. Then NaBH₄ (10 g) was added and the solution was stirred for a further 20 hours at 20° C. Subsequently, CH₃COOH was added to the solution until pH 5.2 and the mixture was stirred for 2 hours at 20° C. The resulting solution was then precipitated by 3,000 mL of isopropanol, the solid was filtered out and dried at reduced pressure. The product was analysed using NMR, DS 54% and Mw 605 kg·mol⁻¹.

### Example 28

### Reduction of Reduced Hyaluronic Acid Using DMTMM and NaBH₄ in Water and Acetonitrile

To a solution of reduced HA (9.0 g) prepared according Example 27 in 1,000 mL of water and 860 mL of acetonitrile, DMTMM (10 g) was added and the mixture was stirred for 24 hours at 20° C. Then NaBH₄ (10 g) was added and the solution was stirred for a further 20 hours at 20° C. Subsequently, CH₃COOH was added to the solution until pH 5.1 and the mixture was stirred for 2 hours at 20° C. The resulting solution was then precipitated by 3,000 mL of isopropanol, the solid was filtered out and dried at reduced pressure. The product was analysed using NMR, DS 62% and SECMALLS, Mw 510 kg·mol⁻¹.

### Example 29

### Hyaluronic Acid Reduction Using DMTMM and NaBH₄ in Water and Acetonitrile

To a solution of HA (10 mg, 1,350 kg·mol⁻¹) in 1 mL of water and 0.86 mL of acetonitrile, DMTMM (15 mg) was added and the mixture was stirred for 24 hours at 20° C. Then NaBH₄ (10 mg) was added and the solution was stirred for a further 20 hours at 20° C. Subsequently, CH₃COOH was added to the solution until pH 5.1 and the mixture was stirred for 2 hours at 20° C. The resulting solution was then precipitated by 3 mL of isopropanol, the solid was filtered out and dried at reduced pressure. The product was analysed using NMR, DS 35% and Mw 715 kg·mol⁻¹.

### Example 30

### Hyaluronic Acid Reduction Using DMTMM and NaBH₄ in Water and Acetonitrile

To a solution of HA (10 mg, 1,350 kg·mol⁻¹) in 1 mL of water and 0.86 mL of acetonitrile, DMTMM (7 mg) was added and the mixture was stirred for 24 hours at 20° C. Then NaBH₄ (10 mg) was added and the solution was stirred for further 20 hours at 20° C. Subsequently, CH₃COOH was added to the solution until pH 5.1 and the mixture was stirred for 2 hours at 20° C. The resulting solution was then precipitated by 3 mL of isopropanol, the solid was filtered out and dried at reduced pressure. The product was analysed using NMR, DS 21% and Mw 898 kg·mol⁻¹.

### Example 31

### Hyaluronic Acid Reduction Using DMTMM and NaBH₄ in Water and Acetonitrile

To a solution of HA (10 mg, 1,350 kg·mol⁻¹) in 1 mL of water and 0.86 mL of acetonitrile, DMTMM (10 mg) was added and the mixture was stirred for 24 hours at 20° C. Then NaBH₄ (1 g) was added (15 mg) and the solution was stirred for further 20 hours at 20° C. Subsequently, CH₃COOH was added to the solution until pH 5.2 and the mixture was stirred for 2 hours at 20° C. The resulting solution was then precipitated by 3 mL of isopropanol, the solid was filtered out and dried at reduced pressure. The product was analysed using NMR, DS 26% and Mw 812 kg·mol⁻¹.

### Example 32

### Hyaluronic Acid Reduction Using DMTMM and NaBH₄ in Water and Acetonitrile

To a solution of HA (10 mg, 1,350 kg·mol⁻¹) in 1 mL of water and 0.86 mL of acetonitrile, DMTMM (10 mg) was added and the mixture was stirred for 24 hours at 20° C. Then NaBH₄ (1 g) was added (5 mg) and the solution was stirred for further 20 hours at 20° C. Subsequently, CH₃COOH was added to the solution until pH 5.2 and the mixture was stirred for 2 hours at 20° C. The resulting solution was then precipitated by 3 mL of isopropanol, the solid was filtered out and dried at reduced pressure. The product was analysed using NMR, DS 25% and Mw 902 kg·mol⁻¹.

### Example 33

### Hyaluronic Acid Reduction Using DMTMM and NaBH₄ in Water and Acetonitrile

To a solution of HA (10 mg, 1,350 kg·mol⁻¹) in 1 mL of water and 0.86 mL of acetonitrile, DMTMM (10 mg) was added and the mixture was stirred for 24 hours at 20° C. Then NaBH₄ (10 mg) was added and the solution was stirred for further 2 hours at 20° C. Subsequently, CH₃COOH was added to the solution until pH 5.5 and the mixture was stirred for 2 hours at 20° C. The resulting solution was then precipitated by 3 mL of isopropanol, the solid was filtered out and dried at reduced pressure. The product was analysed using NMR, DS 30% and Mw 912 kg·mol⁻¹.

### Example 34

### Hyaluronic Acid Reduction Using DMTMM and NaBH₄ in Water and Acetonitrile

To a solution of HA (10 mg, 1,350 kg·mol⁻¹) in 1 mL of water and 0.86 mL of acetonitrile, DMTMM (10 mg) was added and the mixture was stirred for 24 hours at 20° C. Then NaBH₄ (10 mg) was added and the solution was stirred for further 30 hours at 20° C. Subsequently, CH₃COOH was added to the solution until pH 5.1 and the mixture was stirred for 2 hours at 20° C. The resulting solution was then precipitated by 3 mL of isopropanol, the solid was filtered out and dried at reduced pressure. The product was analysed using NMR, DS 27% and Mw 805 kg·mol⁻¹.

### Example 35

### Hyaluronic Acid Reduction Using DMTMM and NaBH₄ in Water and Acetonitrile

To a solution of HA (10 g, 1,800 kg·mol⁻¹) in 1,000 mL of water and 860 mL of acetonitrile, DMTMM (10 g) was added and the mixture was stirred for 24 hours at 20° C. Then NaBH₄ (10 g) was added and the solution was stirred for further 20 hours at 20° C. Subsequently, CH₃COOH was added to the solution until pH 5.1 and the mixture was stirred for 2 hours at 20° C. The resulting solution was then precipitated by 3,000 mL of isopropanol, the solid was filtered out and dried at reduced pressure. The product was analysed using NMR, DS 26% and SECMALLS, Mw 866 kg·mol⁻¹.

### Example 36

### Reduction of Reduced Hyaluronic Acid Using DMTMM and NaBH₄ in Water and Acetonitrile

To a solution of reduced HA (4.5 g) prepared according Example 35 in 500 mL of water and 430 mL of acetonitrile, DMTMM (5 g) was added and the mixture was stirred for 24 hours at 20° C. Then NaBH₄ (5 g) was added and the solution was stirred for further 20 hours at 20° C. Subsequently, CH₃COOH was added to the solution until pH 4.9 and the mixture was stirred for 2 hours at 20° C. The resulting solution was then precipitated by 1,500 mL of isopropanol, the solid was filtered out and dried at reduced pressure. The product was analysed using NMR, DS 54% and Mw 622 kg·mol⁻¹.

### Example 37

### Reduction of Reduced Hyaluronic Acid Using DMTMM and NaBH₄ in Water and Acetonitrile

To a solution of reduced HA (3.6 g) prepared according Example 36 in 500 mL of water and 430 mL of acetonitrile, DMTMM (5 g) was added and the mixture was stirred for 24 hours at 20° C. Then NaBH₄ (5 g) was added and the solution was stirred for further 20 hours at 20° C. Subsequently, CH₃COOH was added to the solution until pH 5.2 and the mixture was stirred for 2 hours at 20° C. The resulting solution was then precipitated by 3,000 mL of isopropanol, the solid was filtered out and dried at reduced pressure. The product was analysed using NMR, DS 60%, Mw 501 kg·mol⁻¹

### Example 38

### Reduction of Reduced Hyaluronic Acid Using DMTMM and NaBH₄ in Water and Acetonitrile

To a solution of reduced HA (2.9 g) prepared according Example 37 in 500 mL of water and 430 mL of acetonitrile, DMTMM (5 g) was added and the mixture was stirred for 24 hours at 20° C. Then NaBH₄ (5 g) was added and the solution was stirred for further 20 hours at 20° C. Subsequently, CH₃COOH was added to the solution until pH 5.1 and the mixture was stirred for 2 hours at 20° C. The resulting solution was then precipitated by 3,000 mL of isopropanol, the solid was filtered out and dried at reduced pressure. The product was analysed using NMR, DS 67% and SECMALLS, Mw 402 kg·mol⁻¹.

### Example 39

### Reduction of Reduced Hyaluronic Acid Using DMTMM and NaBH₄ in Water and Acetonitrile

To a solution of reduced HA (2.2 g) prepared according Example 38 in 300 mL of water and 258 mL of acetonitrile, DMTMM (5 g) was added and the mixture was stirred for 24 hours at 20° C. Then NaBH₄ (5 g) was added and the solution was stirred for further 20 hours at 20° C. Subsequently, CH₃COOH was added to the solution until pH 5.1 and the mixture was stirred for 2 hours at 20° C. The resulting solution was then precipitated by 3,000 mL of isopropanol, the solid was filtered out and dried at reduced pressure. The product was analysed using NMR, DS 75% and SECMALLS, Mw 390 kg·mol⁻¹.

### Example 40

### Hyaluronic Acid Reduction Using DMTMM and NaBH₄ in Water and Acetonitrile

To a solution of HA (10 g, 1,800 kg·mol⁻¹) in 1,000 mL of water and 860 mL of acetonitrile, DMTMM (10 g) was added and the mixture was stirred for 24 hours at 15° C. Then NaBH₄ (10 g) was added and the solution was stirred for further 20 hours at 15° C. Subsequently, CH₃COOH was added to the solution until pH 5.1 and the mixture was stirred for 2 hours at 20° C. The resulting solution was then precipitated by 3,000 mL of isopropanol, the solid was filtered out and dried at reduced pressure. The product was analysed using NMR, DS 25% and SECMALLS, Mw 997 kg·mol⁻¹.

### Example 41

### Hyaluronic Acid Reduction Using DMTMM and NaBH₄ in Water and Acetonitrile

To a solution of HA (0.1 g, 1,350 kg·mol⁻¹) in 12 mL of water and 10.3 mL of acetonitrile, DMTMM (0.1 g) was added and the mixture was stirred for 24 hours at 20° C. Then NaBH₄ (0.1 g) was added and the solution was stirred for further 20 hours at 20° C. Subsequently, CH₃COOH was added to the solution until pH 5.1 and the mixture was stirred for 2 hours at 20° C. The resulting solution was then precipitated by 30 mL of isopropanol, the solid was filtered out and dried at reduced pressure. The product was analysed using NMR, DS 28% and SECMALLS, Mw 971 kg·mol⁻¹.

### Example 42

### Hyaluronic Acid Reduction Using DMTMM and NaBH₄ in Water and Acetonitrile

To a solution of HA (0.1 g, 1,350 kg·mol⁻¹) in 14 mL of water and 12 mL of acetonitrile, DMTMM (0.1 g) was added and the mixture was stirred for 24 hours at 20° C. Then NaBH₄ (0.1 g) was added and the solution was stirred for further 20 hours at 20° C. Subsequently, CH₃COOH was added to the solution until pH 5.1 and the mixture was stirred for 2 hours at 20° C. The resulting solution was then precipitated by 30 mL of isopropanol, the solid was filtered out and dried at reduced pressure. The product was analysed using NMR, DS 27% and SECMALLS, Mw 959 kg·mol⁻¹.

### Example 43

Testing the effect of reduced hyaluronic acid prepared according to Examples 20, 23 and 28 on the viability of 3T3 fibroblasts and cutaneous keratinocytes HaCaT. Both mouse Swiss 3T3 and human HaCaT keratinocytes were cultured in DMEM medium containing 10% FBS (5% CO2, 37° C.). Cytotoxicity was measured via a MTT test. After reaching 80% confluence, the cells were passaged and seeded on a 96-well plate at a density of 3,000 (3T3) or 5,000 (HaCaT) cells per well. After incubation until the next day, the culture medium was replaced with the tested samples (CTRL=culture medium). After 24, 48 and 72 hours of cell treatment, 20 ul of MTT (5 mg/ml) was added, followed by incubation for 2.5 hour. Finally, the culture medium with samples from the plate was tipped out and a lysis solution was added (IPA:DMSO (1:1), Triton X-100 (10%)). After cell lysis, absorption was measured on the Ensight spectrometer (Perkin Elmer) at a wavelength of 570 nm, (690 nm background).

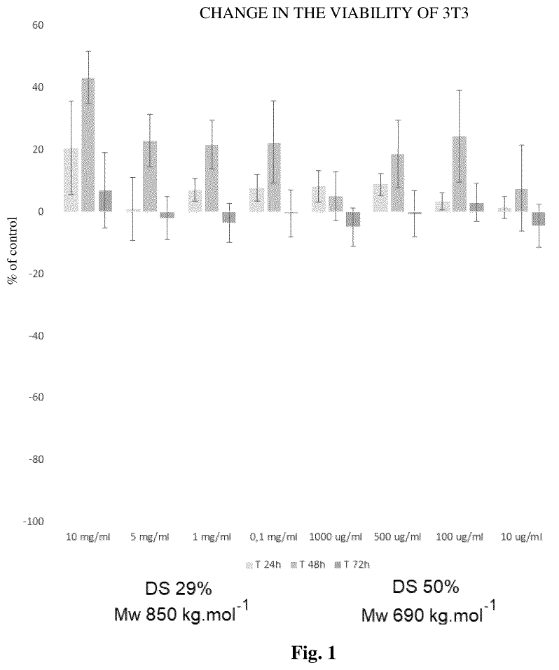

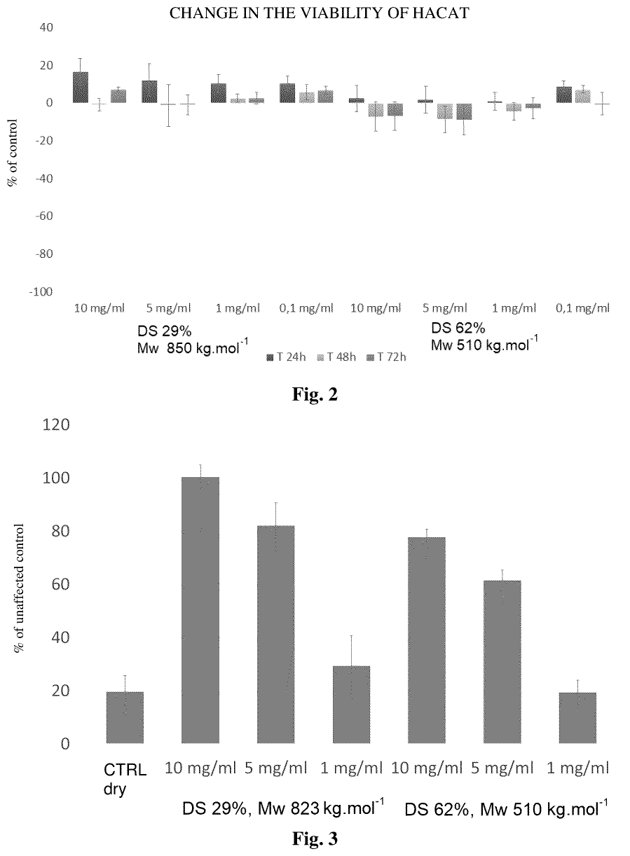

FIGS. 1 and 2 show 3T3, respectively HaCaT viability charts showing the proliferation of cells over time. The graph expresses the change in cell viability relative to control at a given time—that is, the unaffected control corresponds to 0; if the value is positive or negative max. to −20%, this is interpreted as meaning that the substance does not have a cytotoxic effect.

### Example 44

Testing the effect of reduced HA prepared according to Examples 21 and 28 on the viability of HaCaT after drying.

HaCat cells (a line of human epidermal keratinocytes) were seeded on a 48-well plate. After reaching 100% confluence, culture medium was collected and 150 ul of tested samples were applied to the monolayer. The cells were incubated for 30 minutes (37° C., 5% CO₂). Samples were then collected and the lidless plate was inserted into the climate chamber (37° C., 45% humidity) for further 30 minutes. 2 controls were used—cells “dry” without culture medium and standard conditions control with culture medium.

Finally, cell viability was measured using the MTT method. To each well 550 ul of culture medium with MTT (1 mg/ml) was added and cells were incubated for 2.5 hours. After incubation, the cells were lysed with 550 ul of isopropanol:DMSO (1:1%), Triton X-100 (10%). Using a spectrometer (Ensight, Perkin Elmer), absorbance was measured at a reference wavelength of 570 nm and background 690 nm. The results are shown in FIG. 3.

Absorbance values are related to unaffected standard conditions control, which corresponds to 100% viability.

### Example 45

Comparison of Reduced HA Thermal Degradation Prepared According to Examples 20, 22 and 28 with Native HA in Solution

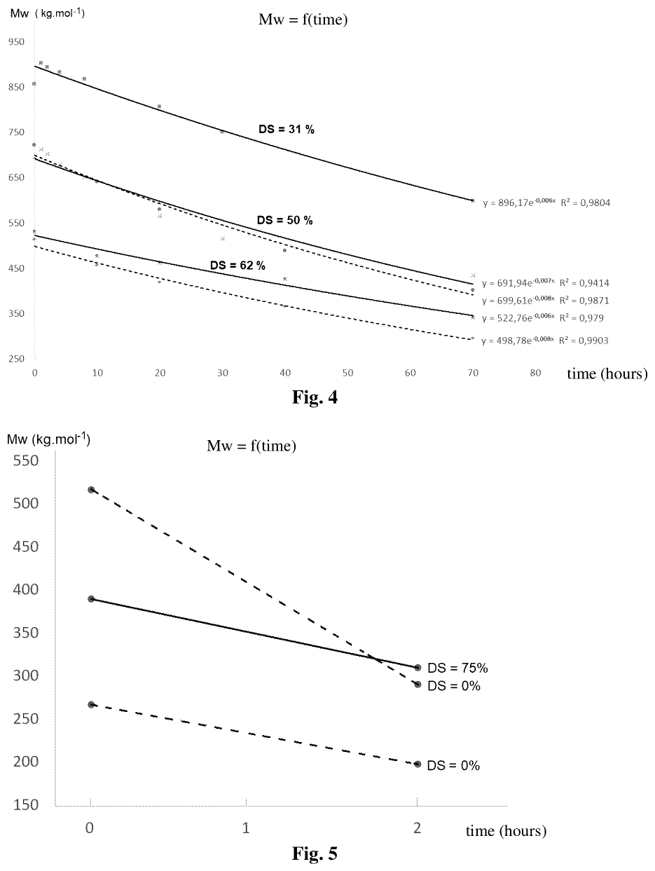

In 40 mL of phosphate buffer solution in water with a pH of 7.4, 200 mg of reduced HA was dissolved. The mixture was stirred at 80° C., with 4 mL of solution collected at specific time intervals (0-70 hours), which were then analysed using the SECMALLS method to determine the molecular weight. The results are shown in FIG. 4.

### Example 46

Thermal Degradation Comparison of Reduced HA Prepared According to Example 39 with Native HA in the Solid Phase

The 50 mg of reduced HA was powdered and heated up at 120° C. for two hours under the atmosphere of the nitrogen gas. In the same way, native hyaluronic acid was heat-stressed (DS=0%, Mw 517 kg·mol⁻¹ and 267 kg·mol⁻¹). Using the SECMALLS method, the molecular weight of the derivative and the unmodified hyaluronic acid were subsequently determined and compared. The results are shown in FIG. 5.

### Example 47

Enzymatic Degradation Comparison of Reduced HA Prepared According to Example 22 and 28 with native HA.

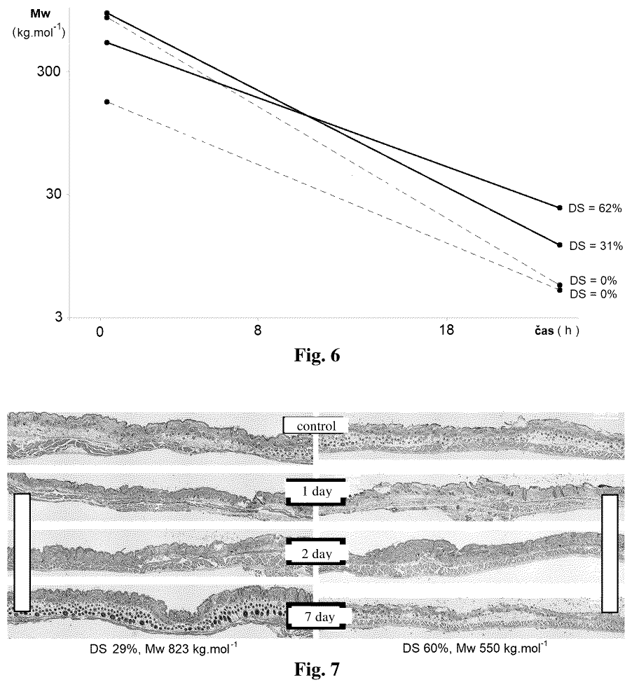

In 0.8 mL of a solution containing 0.01 mol·L⁻¹ acetate buffer and 0.03 mol·L⁻¹ NaCl in D₂O, 8 mg of reduced HA was dissolved. Then acetic acid was added to achieve a pH of 5.3. To this mixture 100 units of the BTH enzyme were added and stirred for 24 hours at 37° C. The final solution was then cooled to 20° C. and measured for NMR. The value of the average molecular weight of the polysaccharide was calculated based on the ratio of the integral of the hydrogen signals of the anomeric end at 5.16 ppm and the integral of the hydrogens of —CH₃ group at 2.0 ppm. The results are shown in FIG. 6.

### Example 48

### Biocompatibility and Safety of Reduced HA Prepared According to Example 21 and 37 in the Subcutaneous Application

The samples of reduced HA (DS 29%, Mw 823 kg·mol⁻¹, DS 60%, Mw 550 kg·mol⁻¹ respectively), were dissolved in saline (0.9% NaCl) to a concentration of 10 mg/mL and sterile filtered (0.22 μm). These solutions were administered subcutaneously at 3 separate sites on the back (20 μl each) to C57Bl/6J strain mice with general anesthesia. After 1, 2 and 7 days, the mice were sacrificed under general anesthetic and skin and tissue samples were collected and fixed in 4% formaldehyde.

Subsequently, histological examination of tissue structure was performed on samples stained with hematoxylin and eosin. Representative histological images are shown in FIG. 7. No pathological changes were observed compared to control (saline) after 1, 2, or 7 days after administration, indicating toxicity, irritability, or other negative effects of reduced HA when administered subcutaneously.

### Example 49

### Preparation of the Composition Based on Reduced HA

The 50 mg of reduced HA prepared according to Example 26 (DS 43% and Mw 729 kg·mol⁻¹) was dissolved in 10 mL of 0.9% NaCl solution in water. The resulting solution was then filtered through a 0.1 to 0.2 μm nitrocellulose filter in sterile conditions. The final solution is suitable for intraarticular application into the synovial cavity.

1-10. (canceled)

11. A reduced hyaluronic acid derivative having the form of a linear chain with a number of native carboxyl groups (—COOH) replaced by primary hydroxyl groups (—CH₂—OH), the hyaluronic acid derivative having a structure according to formula I:
R¹-(A)_{X}-(B)_{Y}—R² (I),
wherein
each A is a structural fragment of the linear chain having a structure according to formula (A): 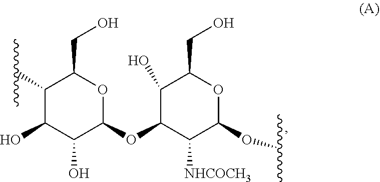
B is a structural fragment of the linear chain having a structure according to formula (B): 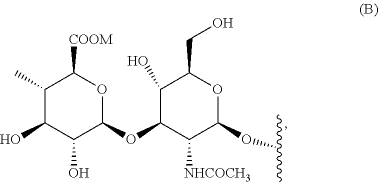
R¹ is a structural fragment of the end of the linear chain and selected from hydroxyl (HO—) and a structure according to formula (C): 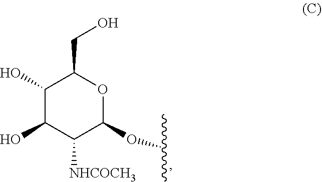
R² is a structural fragment of the end of the linear chain and selected from: 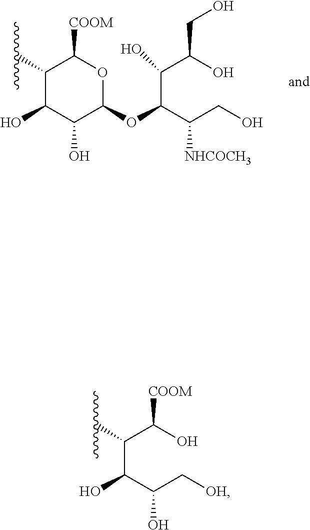
where
each M is hydrogen or an alkali metal cation;
subscript X is an index indicating the number of structural fragments A within the chain, subscript Y is an index indicating the number of structural fragments B within the chain, and each of the structural fragments A and B indicated by subscripts X and Y are randomly distributed in the linear chain;
the sum of X+Y is from 500 to 2,500; and
the ratio of X/Y is from 3/1 to 1/4.

12. A method of preparing the reduced hyaluronic acid derivative of claim 11, said method comprising:
(I) preparing an aqueous solution of an initial hyaluronic acid having a molecular weight of from 400 to 2,200 kg·mol⁻¹ to a concentration of from 0.3 to 2 wt. %;
(II) adding to the aqueous solution acetonitrile in an amount of from 50 to 100% of the volume of the aqueous solution and then 4-(4,6-dimethoxy-1,3,5-triazine-2-yl)-4-methylmorpholinium chloride in an amount of 0.7 to 2.3 wt. equivalent in relation to the initial hyaluronic acid to give a mixture;
(III) reacting the mixture for a time of from 15 to 30 hours at a temperature of from 15 to 25° C., subsequently adding thereto NaBH₄ in an amount of from 0.5 to 2.3 wt. equivalent in relation to the initial hyaluronic acid to give a reduction solution;
(III) stirring the reduction solution for an initial time of from 2 to 30 hours at a temperature of from 15 to 25° C., then adding acetic acid to reach a pH of from 4.5 to 5.5 and stirring for an additional time of from 1.5 to 3 hours at a temperature of from 15 to 25° C. to prepare a reduced hyaluronic acid; and
(IV) isolating the reduced hyaluronic acid from the reduction solution, thereby preparing the reduced hyaluronic acid derivative.

13. The method of claim 12, wherein the initial hyaluronic acid has a molecular weight of from 800 to 1,400 kg·mol⁻¹.

14. The method of claim 12, wherein:
(i) the acetonitrile is added in an amount of 75-90% of the volume of the aqueous solution;
(ii) the 4-(4,6-dimethoxy-1,3,5-triazine-2-yl)-4-methylmorpholinium chloride is added in an amount of from 0.9 to 1.2 wt. equivalent in relation to the initial hyaluronic acid;
(iii) the NaBH₄ is added in the amount of from 0.7 to 1.1 wt. equivalent in relation to the initial hyaluronic acid; or
(iv) any combination of (i)-(iii).

15. The method of claim 12, further comprising subjecting the reduced hyaluronic acid from step (III) to additional reduction by repeating steps (I)-(IV) from 1 to 4 times in sequence, thereby preparing the reduced hyaluronic acid derivative.

16. A composition for cosmetic or medical purposes, comprising the reduced hyaluronic acid derivative of claim 11 in a concentration of from 0.01 to 99 wt. % and at least one additive selected from water, sodium chloride, calcium chloride, glycerol, hyaluronic acid, and chondroitin sulfate.

17. The composition of claim 16, wherein the composition comprises an aqueous solvent and is in the form of a solution or a gel.

18. The composition of claim 16, wherein the composition is further defined as a solid substrate selected from a lyophilisate and a powder.

19. A method of using the reduced hyaluronic acid derivative of claim 11, said method comprising administering a composition comprising the reduced hyaluronic acid derivative and formulated for use in viscosupplementation, anti-wrinkle dermal fillers, fascia fillers, eye drops, or contact lens solutions.

20. A method of preparing an implantable medical device, said method comprising preparing a biocompatible composition comprising the reduced hyaluronic acid derivative of claim 11, and preparing a medical device with the biocompatible composition, thereby preparing implantable medical device.

21. A method of using the composition for cosmetic or medical purposes of claim 16, said method comprising administering the composition for viscosupplementation, anti-wrinkle dermal filling, fascia filling, eye care, or contact lens maintenance.

22. A method of preparing an implantable medical device, said method comprising preparing a biocompatible composition from the composition of claim 16, and preparing a medical device with the biocompatible composition, thereby preparing implantable medical device.
